(19) United States
(12) Patent Application Publication
(10) Pub. No.: US 2008/0139578 A1
(43) Pub. Date: Jun. 12, 2008

(54) Substituted piperizines for the treatment of pain

(76) Inventors: Catherine Abbadie, Summit, NJ (US); Prasun K. Chakravarty, Edison, NJ (US); Joseph L. Duffy, Cranford, NJ (US)

Correspondence Address: MERCK AND CO., INC, P O BOX 2000, RAHWAY, NJ, 07065-0907, US

(21) Appl. No.: 11/982,879

(22) Filed: Nov. 6, 2007

Related U.S. Application Data
(60) Provisional application No. 60/861,879, filed on Nov. 30, 2006.
(60) Provisional application No. 60/918,155, filed on Mar. 15, 2007.
(60) Provisional application No. 60/963,914, filed on Aug. 8, 2007.

(51) Int. Cl. A61K 31/495; A61P 29/00

(52) U.S. Cl. 514/255.01

(57) ABSTRACT

The present invention is directed to the use of substituted piperizine compounds represented by Formula I, for the treatment of pain, including acute pain, chronic pain, cancer pain, visceral pain, inflammatory pain, neuropathic pain, post-herpetic neuralgia, diabetic neuropathy, trigeminal neuralgia, migraine, and fibromyalgia.

This case claims priority under 35 U.S.C. § 119 from U.S. Application Nos. 60/861,879 filed Nov. 30, 2006, Application No. 60/918,155 filed Mar. 15, 2007 and Application No. 60/963,914 filed Aug. 8, 2007.

### FIELD OF THE INVENTION

This invention relates to the use of substituted piperizine compounds for the treatment of a variety of pain conditions, including acute pain, chronic pain, cancer pain, visceral pain, inflammatory pain, allodynia, including itch, neuropathic pain, including post herpetic neuralgia and diabetic neuropathy, trigeminal neuralgia, migraine, fibromyalgia, and other forms of pain, as well as other conditions.

### DESCRIPTION OF THE FIGURES

FIG. 1. Describes Anti-Hyperalgesic Effects of Example 1 Compound in Complete Freund's Adjuvant Model of Inflammatory Pain.

FIG. 2. Describes Anti-Allodynic Effects of Example 1 Compound in Capsaicin-Induced Allodynia Model.

FIG. 3. Describes Anti-Allodynic Effects of Example 1 Compound in Spinal Nerve Ligation (SNL)-Induced Allodynia Model.

### BACKGROUND OF THE INVENTION

The compound I-(3,3-diphenylpropanoyl)piperazine, has previously been described, typically as chemical intermediates, for instance in J. Am. Chem. Soc. 1955, 77, 3142; J. Am. Pharm. Assoc. 1957, 46, 279; United States Patent Application Publication No. US 2006/0084660 A1; United States Patent Application Publication No. US 2004/0259866 A1; United States Patent Application Publication No. US 2004/0147529 A1; United States Patent Application Publication No. US 2004/0034035 A1; U.S. Pat. No. 6,011,035. This compound has not been associated with analgesic activity or the treatment of pain conditions.

### SUMMARY OF THE INVENTION

The present invention is directed to the use of substituted piperizine compounds for the treatment of pain conditions, including acute pain, chronic pain, cancer pain, visceral pain, inflammatory pain, including arthritis, headache, including migraine, trigeminal neuralgia, and cluster headaches, allodynia, including itch, neuropathic pain, including post-herpetic neuralgia and diabetic neuropathy, fibromyalgia, sciatica, and back pain.

This invention further comprises methods for the treatment of pain conditions, including acute pain, chronic pain, cancer pain, visceral pain, inflammatory pain, including arthritis, headache, including migraine, trigeminal neuralgia, and cluster headaches, allodynia, including itch, neuropathic pain, including post-herpetic neuralgia and diabetic neuropathy, fibromyalgia, sciatica, and back pain, by administration of a compound of the present invention

The compounds of the present invention are also useful for the treatment of other conditions, including epilepsy or epilepsy conditions, pruritis, itchiness, and allergic dermatitis.

This invention also provides pharmaceutical compositions comprising a compound of the present invention, either alone, or in combination with one or more therapeutically active compounds, or as a pharmaceutically acceptable prodrug, or as a prodrug in combination with one or more therapeutically active compounds, and a pharmaceutically acceptable carrier.

### DETAILED DESCRIPTION OF THE INVENTION

The invention encompasses compounds represented by Formula I:

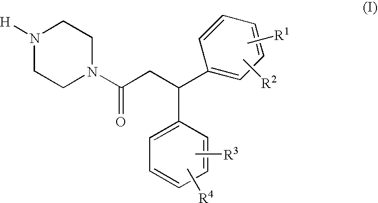

or a pharmaceutically acceptable salt thereof, wherein:
R¹, R², R³, and R⁴ are each independently selected from a group consisting of:
(a) hydrogen,
(b) halogen,
(c) cyano,
(d) hydroxyl,
(e) amino,
(f) C₁-C₈ alkyl,
(g) C₃-C₆ cycloalkyl, and

### (h) O—R⁵,

where each of said alkyl and cycloalkyl is independently optionally substituted with one or more substituents, each substituent selected from halogen, aryl, C₀-C₄ perfluoroalkyl, N(R⁵)₂, —NH(C═O)O—C₁-C₆ alkyl, C₁-C₆ alkyl, C₁-C₆ alkoxy, CN, C₃-C₆ cycloalkyl, OH, —O—C₁-C₄-perfluoroalkyl, C(O)R⁵, C(O)O—R⁵, SO₂R⁵, and heteroaryl, where two adjacent substituents on said aryl or heteroaryl optionally join to form a heterocycle; and
R⁵ is C₁-C₈ alkyl or C₃-C₆ cycloalkyl,
useful for the treatment of pain and other conditions, as described more fully herein. Another sub-embodiment of this invention is realized when the treatment is made in humans.

A preferred embodiment of the present invention includes compound of formula II where R¹, R², R³, and R⁴ are H, that is, the compound:

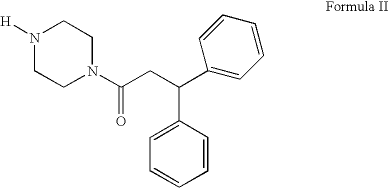

useful for the treatment of pain and other conditions, as described more fully herein. A sub-embodiment of the present invention includes prodrugs of Formula II useful for the treatment of pain and other conditions, as described more fully herein. Another sub-embodiment of this invention is realized when the treatment is made in humans.

Another embodiment of this invention encompasses synthetically produced compounds of formula I, pharmaceutically acceptable salts or prodrugs thereof useful for the treatment of pain and other conditions as described more fully herein. A sub-embodiment of this invention is realized when R¹, R², R³, and R⁴ of Formula I are H. Another sub-embodiment of this invention is realized when the treatment is made in humans.

Another embodiment of this invention relates to use of a pharmaceutical composition comprising a compound of Formula I and pharmaceutically acceptable carrier to treat pain and other conditions as described more fully herein. A sub-embodiment of this invention is realized when R¹, R², R³, and R⁴ of Formula I are H.

Prodrugs are entities structurally related to a biologically active substance (the “parent drug”) which, after administration, separate into the parent drug and a benign byproduct in vivo as the result of some metabolic process, such as enzymatic or chemical hydrolysis of a carboxylic, phosphoric or sulfate ester or reduction or oxidation of a susceptible functionality (see, for example, discussions by (1) A. A. Sinkula and S. H. Yalkowsky, J. Pharm. Sci. 64, 181 (1975); (2) L. A. Svensson, Pharm Weekbl, 122, 245-250 (1987); (3) L. P. Balant, E. Doelker and P. Buri Eur. J. Drug Metab. and Pharmacokinetics, 15, 143-153 (1990); (4) N. Bodor, Drugs of the Future, 6, 165-182 (1981); (5) Design of Biopharmaceutical Properties through Prodrugs and Analogs, E. B. Roche, Ed., American Pharmaceutical Association Academy of Pharmaceutical Sciences, Washington, D.C., (1977); (6) H. Bundgaard Advanced Drug Delivery Reviews, 3, 39-65 (1989)). The advantage of a prodrug may lie in its physical properties, such as enhanced water solubility for parenteral administration compared to the parent drug, or it may enhance absorption from the digestive tract, or it may enhance drug stability for long-term storage. In general, a prodrug possesses less biological activity than its parent drug. For example, a prodrug of a compound of Formula I could include the replacement of the amine hydrogen by a metabolically labile substituent that could include, but is not limited to, alkyl, amide, carbamate, urea, guanidine, cyanoguanidine, imidate, hemi-aminal, aminoketal, amino orthoester, sulfenamide, sulfonamide, and phosphonamide. Specifically excluded from acceptable prodrugs of Formula I are those substituents that would release potentially biologically active or toxic byproducts. Examples of such substituents would include, but are not limited to, diphenylmethy, which would release benzophenone, and alkyl aminonitriles, which would release hydrogen cyanide.

As used herein, “alkyl” as well as other groups having the prefix “alk” such as, for example, alkoxy, alkanoyl, alkenyl, and alkynyl means carbon chains which may be linear or branched or combinations thereof. Examples of alkyl groups include methyl, ethyl, propyl, isopropyl, butyl, sec- and tert-butyl, pentyl, hexyl, and heptyl. “Alkenyl,” “alkynyl” and other like terms include carbon chains containing at least one unsaturated C—C bond.

The term “cycloalkyl” refers to a saturated hydrocarbon containing one ring having a specified number of carbon atoms. Examples of cycloalkyl include cyclopropyl, cyclobutyl, cyclopentyl, and cyclohexyl.

The term “Cₙ” indicates the presence of “n” carbon atoms. For example, “C₁₋₈alkyl” includes alkyls containing 8, 7, 6, 5, 4, 3, 2, or 1 carbon atoms. In certain instances “C₀” is employed (for instance, in the above-listed term “C₀-C₄ perfluoroalkyl” to indicate the presence of no carbon atoms. An alkyl with no carbon atoms is a hydrogen atom substituent when the alkyl is a terminal group and is a direct bond when the alkyl is a bridging group.

The term “alkoxy” as used herein, alone or in combination, includes an alkyl group connected to the oxy connecting atom. The term “alkoxy” also includes alkyl ether groups, where the term ‘alkyl’ is defined above, and ‘ether’ means two alkyl groups with an oxygen atom between them. Examples of suitable alkoxy groups include methoxy, ethoxy, n-propoxy, i-propoxy, n-butoxy, s-butoxy, t-butoxy, methoxymethane (also referred to as ‘dimethyl ether’), and methoxyethane (also referred to as ‘ethyl methyl ether’).

As used herein, “aryl” is intended to mean any stable monocyclic or bicyclic carbon ring of up to 7 members in each ring, wherein at least one ring is aromatic. Examples of such aryl elements include phenyl, napthyl, tetrahydronapthyl, indanyl, or biphenyl.

The term “heterocycle” or “heterocyclic”, as used herein except where noted, represents a stable 5- to 7-membered monocyclic- or stable 8- to 11-membered bicyclic heterocyclic ring system which is either saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Heterocycle includes bicyclic ring systems where one ring is aromatic and the other is not. Examples of heterocyclic groups include, but are not limited to, azetidine, chroman, dihydrofuran, dihydropyran, dioxane, dioxolane, hexahydroazepine, imidazolidine, imidazolidinone, imidazoline, imidazolinone, indoline, isochroman, isoindoline, isothiazoline, isothiazolidine, isoxazoline, isoxazolidine, morpholine, morpholinone, oxazoline, oxazolidine, oxazolidinone, oxetane, 2-oxohexahydroazepin, 2-oxopiperazine, 2-oxopiperidine, 2-oxopyrrolidine, piperazine, piperidine, pyran, pyrazolidine, pyrazoline, pyrrolidine, pyrroline, quinuclidine, tetrahydroquinoline, tetrahydroisoquinolines and oxindoles, tetrahydrofuran, tetrahydropyran, thiamorpholine, thiazoline, thiazolidine, thiomorpholine and N-oxides thereof.

The term “heteroaryl”, as used herein except where noted, represents a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, benzimidazole, benzisothiazole, benzisoxazole, benzofuran, benzothiazole, benzothiophene, benzotriazole, benzoxazole, carboline, cinnoline, furan, furazan, imidazole, indazole, indole, indolizine, isoquinoline, isothiazole, isoxazole, naphthyridine, oxadiazole, oxazole, phthalazine, pteridine, purine, pyran, pyrazine, pyrazole, pyridazine, pyridine, pyrimidine, pyrrole, quinazoline, quinoline, quinoxaline, tetrazole, thiadiazole, thiazole, thiophene, triazine, triazole, and N-oxides thereof.

Examples of heterocycloalkyls include azetidinyl, pyrrolidinyl, piperidinyl, piperazinyl, morpholinyl, tetrahydrofuranyl, imidazolinyl, pyrrolidin-2-one, piperidin-2-one, and thiomorpholinyl.

“Halogen” refers to fluorine, chlorine, bromine and iodine.

The compounds of the present invention contain one or more asymmetric centers and may thus occur as racemates, racemic mixtures, single enantiomers, diastereomeric mixtures, and individual diastereomers.

It will be understood that, as used herein, references to the compounds of structural formula I are meant to also include the pharmaceutically acceptable salts, and also salts that are not pharmaceutically acceptable when they are used as precursors to the free compounds or in other synthetic manipulations.

The compounds of the present invention may be administered in the form of a pharmaceutically acceptable salt. The term “pharmaceutically acceptable salts” refers to salts prepared from pharmaceutically acceptable non-toxic bases or acids.

When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

The pharmaceutical compositions of the present invention comprise compounds of the invention (or pharmaceutically acceptable salts thereof) as an active ingredient, a pharmaceutically acceptable carrier, and optionally one or more additional therapeutic agents or adjuvants. Such additional therapeutic agents can include, for example, i) opiate agonists or antagonists, ii) calcium channel antagonists, iii) 5HT receptor agonists or antagonists, including 5-HT_{1A} agonists or antagonists, 5-HT_{1B/1D} agonists or antagonists and 5-HT_{1A} partial agonists, iv) sodium channel antagonists, v) N-methyl-D-aspartate (NMDA) receptor agonists or antagonists, vi) COX-2 selective inhibitors, vii) neurokinin receptor 1 (NK1) antagonists, viii) non-steroidal anti-inflammatory drugs (NSAID), ix) selective serotonin reuptake inhibitors (SSRI) and/or selective serotonin and norepinephrine reuptake inhibitors (SSNRI), x) tricyclic antidepressant drugs, xi) norepinephrine modulators, xii) lithium, xiii) valproate, xiv) norepinephrine reuptake inhibitors, xv) monoamine oxidase inhibitors (MAOIs), xvi) reversible inhibitors of monoamine oxidase (RIMAs), xvii) alpha-adrenoreceptor antagonists, xviii) atypical anti-depressants, xix) benzodiazepines, xx) corticotropin releasing factor (CRF) antagonists, xxi) neurontin (gabapentin), xxii) sodium channel blockers and xxiii) pregabalin.

The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The present compounds and compositions are useful for the treatment of acute, chronic, visceral, inflammatory and neuropathic pain syndromes. They are useful for the treatment of pain resulting from traumatic nerve injury, nerve compression or entrapment, postherpetic neuralgia, trigeminal neuralgia, and diabetic neuropathy. The present compounds and compositions are also useful for the treatment of chronic lower back pain, phantom limb pain, chronic pelvic pain, neuroma pain, complex regional pain syndrome, chronic arthritic pain and related neuralgias, pain associated with cancer, chemotherapy, HIV and HIV treatment-induced neuropathy, fibromyalgia and headache pain including migraine pain. Compounds of this invention may also be utilized as local anesthetics. Compounds of this invention are useful for the treatment of pruritis, itchiness, and allergic dermatitis. Compounds of this invention are also useful for the treatment of epilepsy and epilepsy conditions.

The subject treated in the present methods is generally a mammal, for example a human being, male or female. The term “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder.

In addition to primates, such as humans, a variety of other mammals can be treated according to the method of the present invention. For instance, mammals including, but not limited to, cows, sheep, goats, horses, dogs, cats guinea pigs, or other bovine, ovine, equine, canine, feline, rodent such as mouse, species can be treated. However, the method can also be practiced in other species, such as avian species (e.g., chickens).

It is understood that compounds of this invention can be administered at prophylactically effective dosage levels to prevent the above-recited conditions and disorders.

Creams, ointments, jellies, solutions, or suspensions containing the instant compounds can be employed for topical use. Mouth washes and gargles are included within the scope of topical use for the purposes of this invention.

Dosage levels from about 0.01 mg/kg to about 140 mg/kg of body weight per day are useful in the treatment of inflammatory and neuropathic pain, or alternatively about 0.5 mg to about 7 g per patient per day. For example, inflammatory pain may be effectively treated by the administration of from about 0.01 mg to about 75 mg of the compound per kilogram of body weight per day, or alternatively about 0.5 mg to about 3.5 g per patient per day. Neuropathic pain may be effectively treated by the administration of from about 0.01 mg to about 125 mg of the compound per kilogram of body weight per day, or alternatively about 0.5 mg to about 5.5 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors. Such patient-related factors include the age, body weight, general health, sex, and diet of the patient. Other factors include the time and route of administration, rate of excretion, drug combination, and the severity of the particular disease undergoing therapy.

In practice, the compounds of the invention, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of the invention, or pharmaceutically acceptable salts thereof, can also be included in pharmaceutical compositions in combination with one or more therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, and disintegrating agents can be used to form oral solid preparations such as powders, capsules and tablets. Because of their ease of administration, tablets and capsules are advantageous oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet advantageously contains from about 0.1 mg to about 500 mg of the active ingredient and each cachet or capsule advantageously containing from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and thus should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, and dusting powder. Further, the compositions can be in a form suitable for use in transdermal devices. These formulations may be prepared, utilizing a compound represented of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid, such as, for example, where the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, and preservatives (including anti-oxidants). Furthermore, other adjuvants can be included to render the formulation isotonic with the blood of the intended recipient. Compositions containing a compound of the invention, or pharmaceutically acceptable salts thereof, can also be prepared in powder or liquid concentrate form.

Further, as described above, the instant compounds can be utilized in combination with one or more therapeutically active compounds. In particular, the inventive compounds can be advantageously used in combination with i) opiate agonists or antagonists, ii) calcium channel antagonists, iii) 5HT receptor agonists or antagonists, including 5-HT_{1A} agonists or antagonists, 5-HT_{1B/1D} agonists or antagonists and 5-HT_{1A} partial agonists, iv) sodium channel antagonists, v) N-methyl-D-aspartate (NMDA) receptor agonists or antagonists, vi) COX-2 selective inhibitors, vii) neurokinin receptor 1 (NK1) antagonists, viii) non-steroidal anti-inflammatory drugs (NSAID), ix) selective serotonin reuptake inhibitors (SSRI) and/or selective serotonin and norepinephrine reuptake inhibitors (SSNRI), x) tricyclic antidepressant drugs, xi) norepinephrine modulators, xii) lithium, xiii) valproate, xiv) norepinephrine reuptake inhibitors, xv) monoamine oxidase inhibitors (MAOIs), xvi) reversible inhibitors of monoamine oxidase (RIMAs), xvii) alpha-adrenoreceptor antagonists, xviii) atypical anti-depressants, xix) benzodiazepines, xx) corticotropin releasing factor (CRF) antagonists, xxi) neurontin (gabapentin), xxii) sodium channel blockers and xxiii) pregabalin.

The present compounds can be prepared according to the general Scheme provided below as well as the procedure provided in the Example. The following Scheme and Examples further describe, but do not limit, the scope of the invention.

Unless specifically stated otherwise, the experimental procedures were performed under the following conditions: All operations were carried out at room or ambient temperature; that is, at a temperature in the range of 18-25° C. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) or by high-pressure liquid chromatography-mass spectrometry (HPLC-MS), and reaction times are given for illustration only. The structure and purity of all final products were assured by at least one of the following techniques: TLC, HPLC, mass spectrometry, nuclear magnetic resonance (NMR) spectrometry or microanalytical data. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. Broad; etc. Chemical symbols have their usual meanings; the following abbreviations are used: mL (milliliters), g (gram(s)), mg (milligrams(s)), mol (moles), mmol (millimoles), eq (equivalent(s)).

### Methods of Synthesis

Compounds of the present invention can be prepared according to the Scheme provided below as well as the procedures provided in the Examples. The substituents are the same as in the above Formulas except where defined otherwise or otherwise apparent to the ordinary skilled artisan.

The novel compounds of the present invention can be readily synthesized using techniques known to those skilled in the art, such as those described, for example, in Advanced Organic Chemistry, March, 5^{th} Ed., John Wiley and Sons, New York, N.Y., 2001; Advanced Organic Chemistry, Carey and Sundberg, Vol. A and B, 3^{rd} Ed., Plenum Press, Inc., New York, N.Y., 1990; Protective groups in Organic Synthesis, Green and Wuts, 2^{nd} Ed., John Wiley and Sons, New York, N.Y., 1991; Comprehensive Organic Transformations, Larock, VCH Publishers, Inc., New York, N.Y., 1988; Handbook of Heterocyclic Chemistry, Katritzky and Pozharskii, 2^{nd} Ed., Pergamon, New York, N.Y., 2000 and references cited therein. The starting materials for the present compounds may be prepared using standard synthetic transformations of chemical precursors that are readily available from commercial sources, including Aldrich Chemical Co. (Milwaukee, Wis.); Sigma Chemical Co. (St. Louis, Mo.); Lancaster Synthesis (Windham, N.H.); Ryan Scientific (Columbia, S.C.); Maybridge (Cornwall, UK); Matrix Scientific (Columbia, S.C.); Arcos, (Pittsburgh, Pa.) and Trans World Chemicals (Rockville, Md.).

The procedures described herein for synthesizing the compounds may include one or more steps of protecting group manipulations and of purification, such as, recrystallization, distillation, column chromatography, flash chromatography, thin-layer chromatography (TLC), radial chromatography and high-pressure chromatography (HPLC). The products can be characterized using various techniques well known in the chemical arts, including proton and carbon-13 nuclear magnetic resonance (¹H and ¹³C NMR), infrared and ultraviolet spectroscopy (IR and UV), X-ray crystallography, elemental analysis and HPLC and mass spectrometry (HPLC-MS). Methods of protecting group manipulation, purification, structure identification and quantification are well known to one skilled in the art of chemical synthesis.

Appropriate solvents are those which will at least partially dissolve one or all of the reactants and will not adversely interact with either the reactants or the product. Suitable solvents are aromatic hydrocarbons (e.g., toluene, xylenes), halogenated solvents (e.g., methylene chloride, chloroform, carbontetrachloride, chlorobenzenes), ethers (e.g., diethyl ether, diisopropylether, tert-butyl methyl ether, diglyme, tetrahydrofuran, dioxane, anisole), nitriles (e.g., acetonitrile, propionitrile), ketones (e.g., 2-butanone, dithyl ketone, tert-butyl methyl ketone), alcohols (e.g., methanol, ethanol, n-propanol, iso-propanol, n-butanol, t-butanol), N,N-dimethyl formamide (DMF), dimethylsulfoxide (DMSO) and water. Mixtures of two or more solvents can also be used. Suitable bases are, generally, alkali metal hydroxides, alkaline earth metal hydroxides such as lithium hydroxide, sodium hydroxide, potassium hydroxide, barium hydroxide, and calcium hydroxide; alkali metal hydrides and alkaline earth metal hydrides such as lithium hydride, sodium hydride, potassium hydride and calcium hydride; alkali metal amides such as lithium amide, sodium amide and potassium amide; alkali metal carbonates and alkaline earth metal carbonates such as lithium carbonate, sodium carbonate, cesium carbonate, sodium hydrogen carbonate, and cesium hydrogen carbonate; alkali metal alkoxides and alkaline earth metal alkoxides such as sodium methoxide, sodium ethoxide, potassium tert-butoxide and magnesium ethoxide; alkali metal alkyls such as methyllithium, n-butyllithium, sec-butyllithium, t-bultyllithium, phenyllithium, alkyl magnesium halides, organic bases such as trimethylamine, triethylamine, triisopropylamine, N,N-diisopropylethylamine, piperidine, N-methyl piperidine, morpholine, N-methyl morpholine, pyridine, collidines, lutidines, and 4-dimethylaminopyridine; and bicyclic amines such as DBU and DABCO.

As described previously, in preparing the compositions for oral dosage form, any of the usual pharmaceutical media can be employed. For example, in the case of oral liquid preparations such as suspensions, elixirs and solutions, water, glycols, oils, alcohols, flavoring agents, preservatives, coloring agents and the like may be used; or in the case of oral solid preparations such as powders, capsules and tablets, carriers such as starches, sugars, microcrystalline cellulose, diluents, granulating agents, lubricants, binders, disintegrating agents, and the like may be included. Because of their ease of administration, tablets and capsules represent the most advantageous oral dosage unit form in which solid pharmaceutical carriers are employed. If desired, tablets may be coated by standard aqueous or nonaqueous techniques. In addition to the common dosage forms set out above, controlled release means and/or delivery devices may also be used in administering the instant compounds and compositions.

It is understood that the functional groups present in compounds described in the Schemes below can be further manipulated, when appropriate, using the standard functional group transformation techniques available to those skilled in the art, to provide desired compounds described in this invention.

It is also understood that compounds listed in the Schemes and Tables below that contain one or more stereocenters may be prepared as single enantiomers or diastereomers, or as mixtures containing two or more enantiomers or diastereomers in any proportion.

Other variations or modifications, which will be obvious to those skilled in the art, are within the scope and teachings of this invention. This invention is not to be limited except as set forth in the following claims.

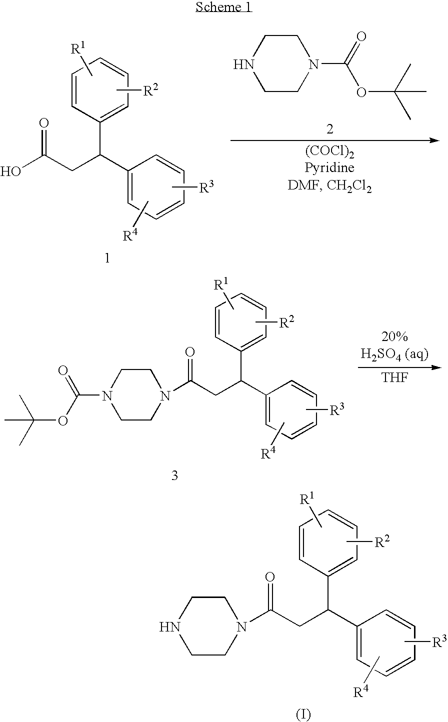

The compounds of the present invention may be prepared as illustrated in Scheme 1. The appropriately substituted diphenylpropionic acid 1, which may be either commercially available or prepared by those skilled in the art, may be treated with an activating agent such as oxalyl chloride in the presence of a base such as pyridine in an appropriate solvent or solvent mixture such as dimethylformamide and dichloromethane. The resulting diphenylpropionic acid chloride intermediate solution may then be diluted with a solvent such as dichloromethane, and an appropriately substituted amine such as N-Boc-piperazine 2 may be added, to afford a carbamate-protected intermediate such as 3. The carbamate group may be removed with an acid such as aqueous H₂SO₄ in tetrahydrofuran, to afford compounds represented by I.

### EXAMPLE 1

### Preparation of 1-(3,3-diphenylpropanoyl)piperazine

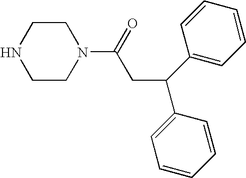

A solution of diphenylpropionic acid (2.0 g) in a 1:1 mixture of dimethylformamide and dichloromethane (10 mL) was added to a solution of oxalyl chloride (4.8 mL) in a 1:1 mixture of dimethylformamide and dichloromethane (10 mL) at 0° C. After 30 minutes, N-Boc-piperazine (1.96 g) was added, followed by pyridine (1.4 mL). The mixture was stirred at ambient temperature for 18 h, then diluted with saturated aqueous NaHCO₃ and extracted with ethyl acetate and concentrated. The residue was purified by silica gel chromatography using a gradient of 1:7 to 1:4 ethyl acetate in hexanes, affording the N-Boc protected piperazine amide as a white solid. This material was dissolved in 20 mL of tetrahydrofuran, and to this solution was added 20% aqueous H₂SO₄ (10 mL). The mixture was heated to 70° C. for 2 hours, then cooled to ambient temperature and adjusted to alkaline pH with solid KOH. The mixture was extracted with ethyl acetate, and the organic layer was dried over Na₂SO₄ and concentrated to afford the title compound as a white solid. ¹H NMR (CDCl₃): 7.29 (m, 6H), 7.23 (m, 4H), 4.61 (t, J=7.1 Hz, 1H), 3.85 (s, 2H), 3.65 (s, 2H), 3.06 (d, J=7.3 Hz, 2H), 2.97 (s, 2H), 2.60 (s, 2H). MS m/e 295 (M+1)⁺.

### EXAMPLE 2

### In Vivo Assay

### Rodent CFA Model

Male Sprague Dawley rats (300-400 gm) were administered 200 microl CFA (Complete Freund's Adjuvant) three days prior to the study. CFA is mycobacterium tuberculosis suspended in saline (1:1; Sigma) to form an emulsion that contains 0.5 mg mycobacterium/ml. The CFA was injected into the plantar area of the left hind paw.

Rats were fasted the night before the study only for oral administration of compounds. On the morning of test day using a Ugo Basile apparatus, 2 baseline samples were taken 1 hour apart. The rat was wrapped in a towel. Its paw was placed over a ball bearing and under the pressure device. A foot pedal was depressed to apply constant linear pressure. Pressure was stopped when the rat withdrew its paw, vocalized, or struggled. The right paw was then tested. Rats were then dosed with the Example 1 compound and tested at predetermined time points.

Compounds were prepared in Imwitor:Tween 80 (1:1) and were dosed in a volume of 2 ml/kg. Percent maximal possible effect (% MPE) was calculated as: (post-treatment−pre-treatment)/(pre-injury threshold−pre-treatment)×100. The effect of treatment was determined by one-way ANOVA Repeated Measures Friedman Test with a Dunn's post test.

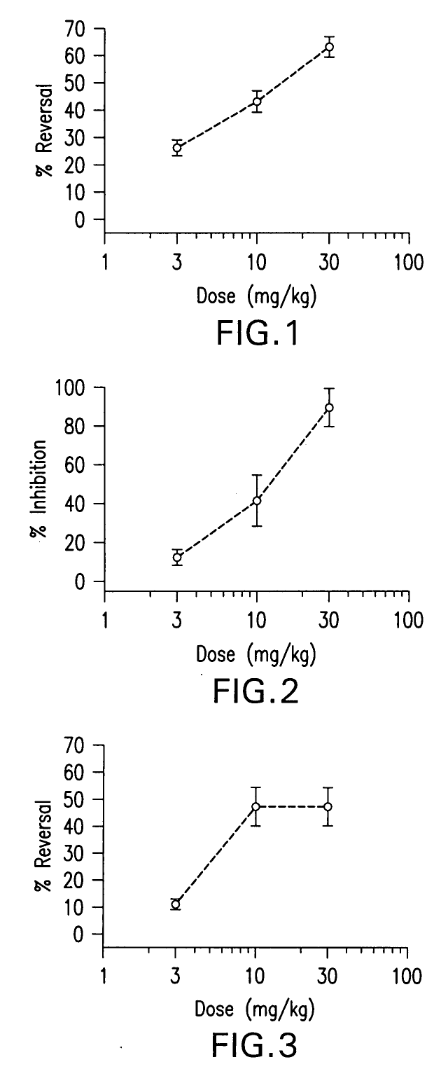

In the CFA model, the compound in example 1 dose-dependently reversed mechanical hyperalgesia. One hour post-dose, example 1 induced 26, 43 and 63% reversal of hyperalgesia at 3, 10 and 30 mg/kg P.O. respectively. At 3 hr post-dose, % reversals were 17, 39 and 44% at 3, 10 and 30 mg/kg P.O. respectively, as illustrated in FIG. 1.

### EXAMPLE 3

### In Vivo Assay

### Capsaicin-Induced Allodynia Model

Male Sprague-Dawley (Charles River) rats were habituated to the von Frey stands for 4 hr the day prior to the testing day. Tactile allodynia was assessed using calibrated von Frey filaments (up-down method) before and 30, 60 and 120 min post capsaicin injection. Capsaicin (30 μg in 10 μl) was diluted in Ethanol:Tween80:Saline (20:7:73) and administered into the rat left paw. The test compound is administered P.O. at 3, 10 or 30 mg/kg (4 ml/kg) 30 min prior to capsaicin administration. Data are expressed as % inhibition=(post-treatment−post-vehicle)/(pre-capsaicin threshold−post-vehicle)×100. Statistic analysis: 2-way ANOVA (time and dose) and Bonferroni post-hoc analysis comparing each dose to vehicle.

In the capsaicin-induced allodynia model, the compound in example 1 dose-dependently blocked capsaicin-induced allodynia: maximal percent inhibition of allodynia was 33, 46 and 89%, respectively when administered at 3, 10 and 30 mg/kg P.O. 30 min before capsaicin challenge, as illustrated in FIG. 2.

### EXAMPLE 4

### In Vivo Assay

### Spared Nerve Ligation (SNL) Model

For the neuropathic pain model, rats (Sprague-Dawley, Harlan) were anesthetized with isoflurane and were placed on a heating pad. Using aseptic techniques, the L5 spinal nerve was exposed and ligated. Muscle and skin were closed with 4-0 Polydiaxone and wound clips, respectively. Animals were tested for allodynia 4 weeks post SNL surgery. Only rats that developed allodynia as defined by a significant decrease in their mechanical threshold using von Frey filaments, were used for evaluating the compound. Tactile allodynia was assessed with calibrated von Frey filaments (Stoelting Co), using an up-down paradigm before and at 2 hr and 44 hr post-compound administration. Data are expressed as % reversal=(post-treatment−post-vehicle)/(pre-SNL threshold−post-vehicle)×100.

In the SNL-induced allodynia model, the compound in Example 1 significantly reversed SNL-induced allodynia: maximal percent reversal of allodynia was 11, 47 and 47%, respectively when administered at 3, 10 and 30 mg/kg P.O. as illustrated in FIG. 3.

What is claimed is:

1. A method for treating pain in a patient in need thereof, said method comprising administering to said patient a therapeutically effective amount of a compound according to Formula I: 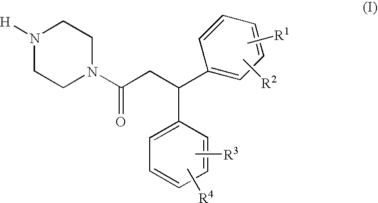
or a pharmaceutically acceptable salt thereof, wherein:
R¹, R², R³, and R⁴ are each independently selected from a group consisting of:
(a) hydrogen,
(b) halogen,
(c) cyano,
(d) hydroxyl,
(e) amino,
(f) C₁-C₈ alkyl,
(g) C₃-C₆ cycloalkyl, and
(h) O—R⁵,
where each of said alkyl and cycloalkyl is independently optionally substituted with one or more substituents, each substituent selected from halogen, aryl, C₀-C₄ perfluoroalkyl, N(R⁵)₂, —NH(C═O)O—C₁-C₆ alkyl, C₁-C₆ alkyl, C₁-C₆ alkoxy, CN, C₃-C₆ cycloalkyl, OH, —O—C₁-C₄-perfluoroalkyl, C(O)R⁵, C(O)O—R⁵, SO₂R⁵, and heteroaryl, where two adjacent substituents on said aryl or heteroaryl optionally join to form a heterocycle; and
R⁵ is C₁-C₈ alkyl or C₃-C₆ cycloalkyl.

2. The method of claim 1, wherein the form of pain treated is selected from acute pain, chronic pain, cancer pain, visceral pain, inflammatory pain, including arthritis pain, headache pain including migraine, trigeminal neuralgia and cluster headaches, allodynia, including itch, neuropathic pain, including post-herpetic neuralgia and diabetic neuropathy, fibromyalgia, sciatica and back pain.

3. The method of claim 1, wherein each of R¹, R², R³, and R⁴ is hydrogen.

4. The method of claim 1 wherein the compound of formula I is synthetically produced.

5. The method of claim 1 for treating humans.

6. A method for treating pain in a patient in need thereof, said method comprising administering to said patient a therapeutically effective amount of the compound: 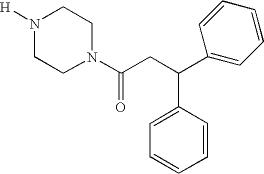
or a pharmaceutically acceptable salt.

7. The method of claim 6 wherein the compound of Formula I is a prodrug that does not liberate benzophenone or hydrogen cyanide as metabolic byproducts.

8. The method of claim 6 wherein the compound of formula I is synthetically produced.

9. The method of claim 6 for treating humans.

10. The method of claim 6, wherein the form of pain treated is selected from acute pain, chronic pain, cancer pain, visceral pain, inflammatory pain, including arthritis pain, headache pain including migraine, trigeminal neuralgia and cluster headaches, allodynia, including itch, neuropathic pain, including post-herpetic neuralgia and diabetic neuropathy, fibromyalgia, sciatica and back pain.

11. A method for treating pain in a patient in need thereof, said method comprising the co-administration, to a person in need of such treatment, of a therapeutically effective amount of the compound: 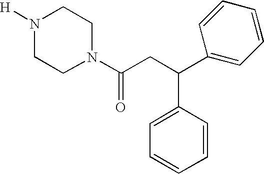
or a pharmaceutically acceptable salt thereof, and a therapeutically effective amount of a second agent selected from, analgesics, anti-inflammatory agents, serotonin agonists and anticonvulsants.
